(19) United States
(12) Patent Application Publication
(10) Pub. No.: US 2015/0343219 A1
(43) Pub. Date: Dec. 3, 2015

(54) SYSTEM AND METHOD TO INITIALIZE THERAPEUTIC TREATMENT FOR MONITORED PHYSIOLOGY DATA

(71) Applicant: Will Rosellini, Dallas, TX (US)

(72) Inventor: Will Rosellini, Dallas, TX (US)

(73) Assignee: Remote Biomedical LLC, Dallas, TX (US)

(21) Appl. No.: 14/290,439

(22) Filed: May 29, 2014

(51) Int. Cl. A61N 1/36; G06F 19/00; A61N 1/372

(52) U.S. Cl. CPC A61N 1/36053; A61N 1/37247; A61N 1/37264; G06F 19/322

(57) ABSTRACT

Disclosed is a system and method for initializing a therapeutic treatment for normalizing a high-priority physiology data by determining the criticality of the patient. The method monitors the physiology data of the patient and determines the criticality of the patient's condition based on the priority computed for the monitored physiology data. Further, the method initiates a therapeutic treatment to normalize the high-priority monitored physiology data by allowing the user to provide input values.

### BACKGROUND

The present invention relates to initializing therapeutic treatment and more particularly to normalizing the priority value computed for one or more monitored physiology data by initializing one or more therapeutic treatment based on one or more input values.

### SUMMARY

The present invention is a method for initializing one or more therapeutic treatments for normalizing a high priority value monitored physiology data, wherein the method comprises of computing a priority value for the monitored physiology data received at the Health Insurance Portability and Accountability Act (HIPAA) server. Further, the method determines the monitored physiology data that has high priority value and allows the user to provide one or more input for initializing the therapeutic treatment for normalizing the high priority value of the monitored physiology data.

Other objects and advantages of the embodiments herein will become readily apparent from the following detailed description taken in conjunction with the accompanying drawings.

### BRIEF DESCRIPTION OF THE DRAWING(S)

FIG. 1, according to an embodiment of the present invention, is an overview of the system for initializing therapeutic treatment based on the monitored physiology data.

FIG. 2, according to an embodiment of the present invention, is an illustration of various modules the system comprises for initializing therapeutic treatment based on the monitored physiology data.

FIG. 3, according to an embodiment of the present invention, is an illustration of a flow-chart that explains the process of normalizing the priority value of the monitored physiology data by initializing the therapeutic treatment.

FIG. 4, according to an embodiment of the present invention, is an illustration of an example to initialize therapeutic treatment for normalizing the priority value computed for the monitored physiology data.

FIG. 5, according to an embodiment of the present invention, is an illustration of a flow-chart that explains the process of computing the priority value of the monitored physiology data.

FIG. 6, according to an embodiment of the present invention, is an illustration of an example with an input provided for initializing therapeutic treatment.

### FIGURES

### Reference Numerals

100—System for initializing therapeutic treatment
101—Network within which the system works
102—Vagus Nerve Stimulation (VNS) polling system
103—Congestive Heart Failure (CHF) monitoring system
104—System for storing or upgrading monitored physiology data on the cloud storage
105—Input provided for initializing therapeutic treatment

### DETAILED DESCRIPTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

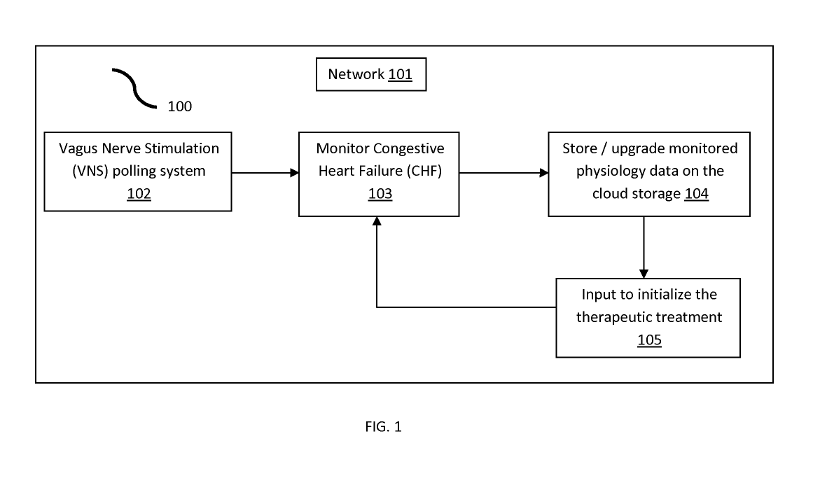

Referring to FIG. 1, the system 100 for initializing therapeutic treatment based on the monitored physiology data comprises of monitoring physiology data using the Vagus Nerve Stimulation (VNS) polling system for monitoring Congestive Heart Failure (CHF). The method computes the priority value for the monitored physiology data. Further, as the priority value is determined to be high for the monitored physiology data, an input value is provided to initialize the therapeutic treatment. After providing the therapeutic treatment, the method allows the Congestive Heart Failure (CHF) monitoring system to monitor the treated physiology data and ensures that the high priority value monitored for the physiology data is normalized.

In an embodiment, the electronic device 100 can be a mobile device, a smart phone device, a lap top, a personal computer or the like that is capable of displaying one or more messages on the screen and highlighting the selected message.

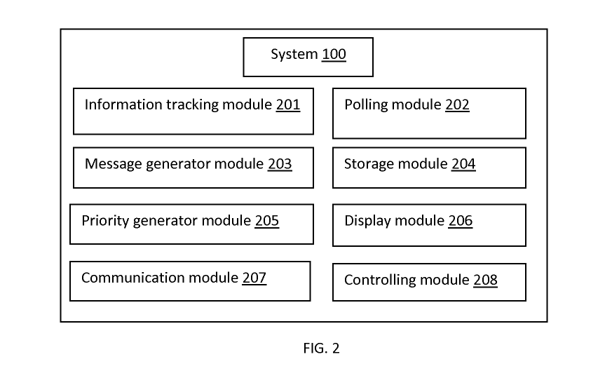

Referring to FIG. 2, the system 100 that implements the proposed invention comprises of the following modules: Information tracking module 201, a Polling module 202, a Message generator module 203, a Storage module 204, a Priority generator module 205, a Display module 206, a Communication module 207, and a Controlling module 208. The Information tracking module 201 is configured to track information about the patient's condition.

The Polling module 202 is configured to poll for the Congestive Heart Failure (CHF) by monitoring the physiology data.

In an embodiment, the Vagus Nerve Stimulation (VNS) polling system 102 is configured to poll for the CHF by monitoring the physiology data of the patient.

The Message generator module 203 is configured to generate message for the patient based on the monitored physiology data. The generated message is displayed on the electronic device 100 as a warning message to the patient about his/her condition. The Storage module 204 is configured to store/upgrade the monitored physiology data along with the priority value computed for the monitored physiology data.

In an embodiment, the monitored physiology data along with the priority value associated with the monitored physiology data can be stored/upgraded in the cloud database or at the Health Insurance Portability and Accountability Act (HIPAA) server.

The Priority generator module 205 is configured to compute/determine priority for the monitored physiology data and is stored in the Storage module 204. The Display module 206 is configured to display the warning message to the patient based on the priority value associated with the monitored physiology data. Further, the Display module 206 is configured to accept the input value provided by the user for initializing the therapeutic treatment.

The Communication module 207 is configured to allow various components in the system to communicate with each other. The Controlling module 208 is configured to control various activities performed within the system 100.

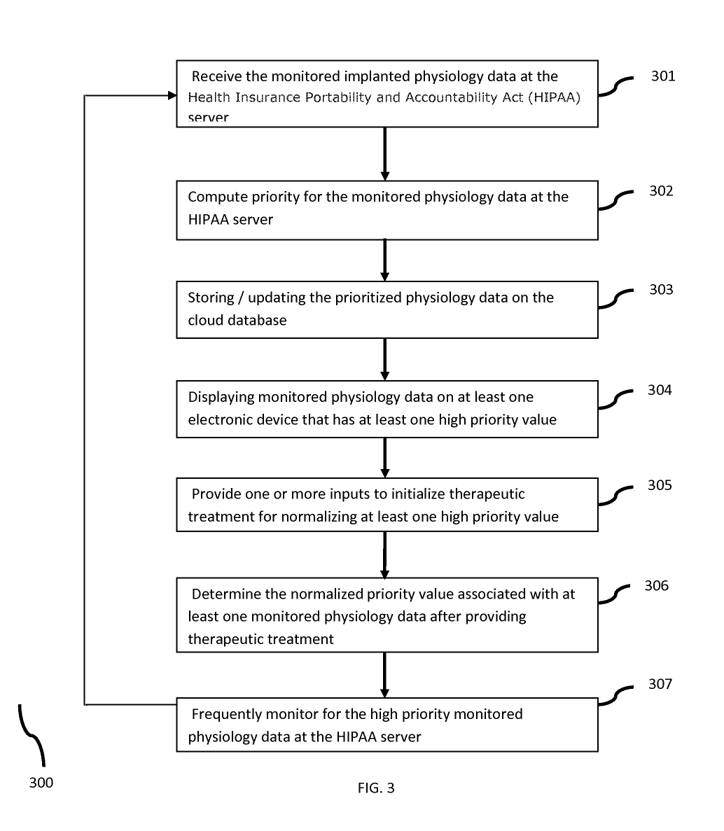

Referring to FIG. 3, the method 300 allows the user to normalize the high priority physiology data by monitoring the physiology data of the patient. Initially, at step 301 as the user monitors the implanted physiology data of the patient, the monitored implanted physiology data is received at the HIPAA server. In an embodiment, the Controlling module 208 is configured to receive the implanted physiology data of the patient at the HIPAA server. At step 302, priority for the monitored physiology data is computed at the HIPAA server. In an embodiment, the Priority generator module 205 is configured to compute/determine priority for the monitored physiology data at the HIPAA server. At step 303, the computed priority of the monitored physiology data is stored/upgraded at the HIPAA server. In an embodiment, the Controlling module 208 stores/upgrades the computed prioritized monitored physiology data at the HIPAA server. At step 304, the monitored physiology data that has a high-priority value is displayed to the patient on the electronic device as a warning message. In an embodiment, the Message generator module 203 is configured to generate a warning message from the high priority monitored physiology data and the Display module 206 is configured to display the generated warning message to the patient. At step 305, based on the warning message displayed on the electronic device, the user can provide inputs to normalize the high priority monitored physiology data. Further, based on these inputs the therapeutic treatment is initialized. In an embodiment, the Display module 206 is configured to receive the inputs to normalize the high priority monitored physiology data and the Controlling module 208 is configured to initialize the therapeutic treatment based on the received input. At step 306, the method 300 monitors the high priority monitored physiology data until the high priority is normalized by providing the therapeutic treatment for the high priority monitored physiology data.

In an embodiment, the therapeutic treatment can be continued until the priority of the monitored physiology data is normalized.

In an embodiment, the Information tracking module 201 is configured to track the high priority monitored physiology data of the patient based on the recomputed priority value of the monitored physiology data after providing the therapeutic treatment.

At step 307, the method 300 frequently monitors for the high priority monitored physiology data at the HIPAA server. In an embodiment, the Controlling module 208 frequently monitors for the high priority monitored physiology data at the HIPAA server.

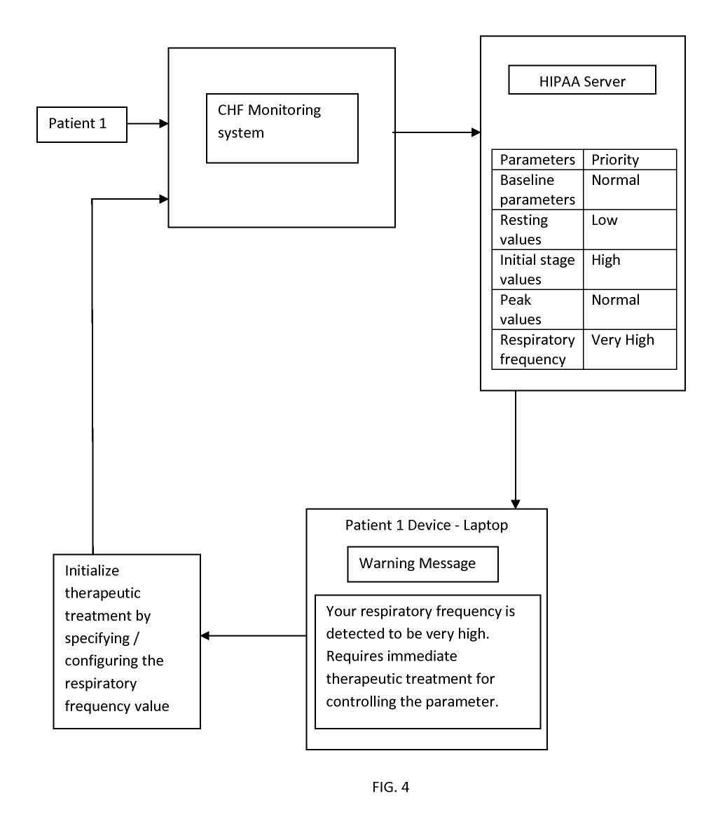

Referring to FIG. 4, when a patient condition is monitored using a monitoring system, one or more physiology parameter values are determined along with the weightage assigned to each of the monitored physiology parameter values. Further, the monitored physiology parameter values along with the priority range are stored on the HIPAA Server. The high-priority monitored physiology parameter is displayed to the patient as a warning message. In the current example, the respiratory frequency of Patient 1 is determined to be associated with a high-priority. Hence, a warning message regarding the respiratory frequency of Patient 1 is displayed on the patient's laptop. Based on this warning message, a therapeutic treatment can be initiated for the patient by providing the input value to normalize the respiratory frequency priority value. Further, as the therapeutic treatment is provided to the patient, the monitoring system continuously monitors the treated parameter to determine if the high-priority value associated with the treated parameter is normalized in the patient.

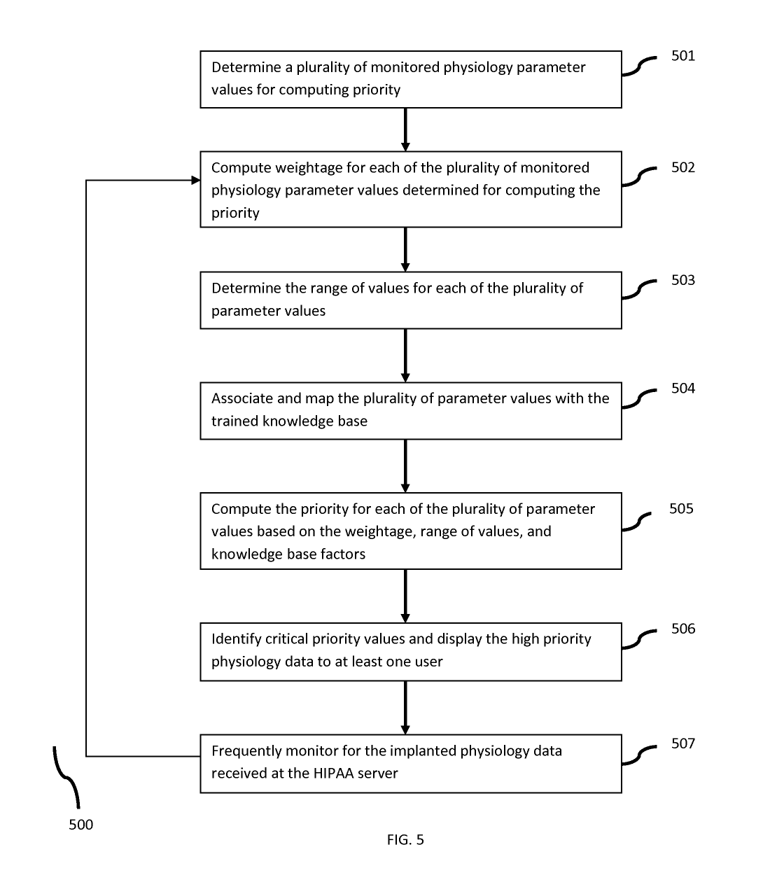

Referring to FIG. 5, the method 500 computes the priority value for the monitored physiology data by determining the weightage, mapping the observed values with the standard range of values in accordance with the determined weightage, and associating the determined weightage and the observed values with the stored knowledge base. Further, the criticality of the patient's condition can be determined based on the priority determined for the monitored physiology data. Initially at step 501, as the monitored physiology data is received at the HIPAA server, a plurality of parameters will be considered to compute priority.

In an embodiment, the Controlling module 208 is configured to select a plurality of parameters for computing the priority. For example, the Controlling module 208 can be configured to select the Baseline parameters and the Resting value parameters for patient 1 to compute the priority. At step 502, each of the plurality of parameters that are selected will be assigned with different weightages. In an embodiment, the Controlling module 208 is configured to assign weightage to each of the plurality of parameters selected for determining the priority. For example, the Controlling module 208 can be configured to assign a high weightage (weightage 1) to the Baseline parameters as compared to the weightage (weightage 5) assigned to the Resting value parameters. At step 503, determine the observed range of values for each of the plurality of parameters after determining the weightage for each of the plurality of parameters. In an embodiment, the Controlling module 208 is configured determine the observed range of values for each of the plurality of parameters. For example, the observed range of values for each of the plurality of parameters includes but not limited to: 64, 24/8, 176, 78. At step 504, the observed range of values and the weightage of each of the plurality of parameters are associated and mapped with an existing knowledge base to compute the priority for the selected plurality of parameters. At step 505, priority value for each of the plurality of parameters is computed. In an embodiment, the Priority generator module 205 is configured to compute the priority for the selected plurality of parameters. At step 506, based on the priority computed for each of the plurality of parameters, criticality of the patient's condition is determined and a warning message is displayed to the patient based on the condition. In an embodiment, the Information tracking module 201 is configured to determine the criticality of the patient's condition and the Message generator module 203 is configured to generate a warning message to be displayed to the patient. At step 507, the implanted physiology data is monitored and the monitored data is received at the HIPAA server. In an embodiment, the Controlling module 208 is configured to monitor the received implanted physiology data at the HIPAA server.

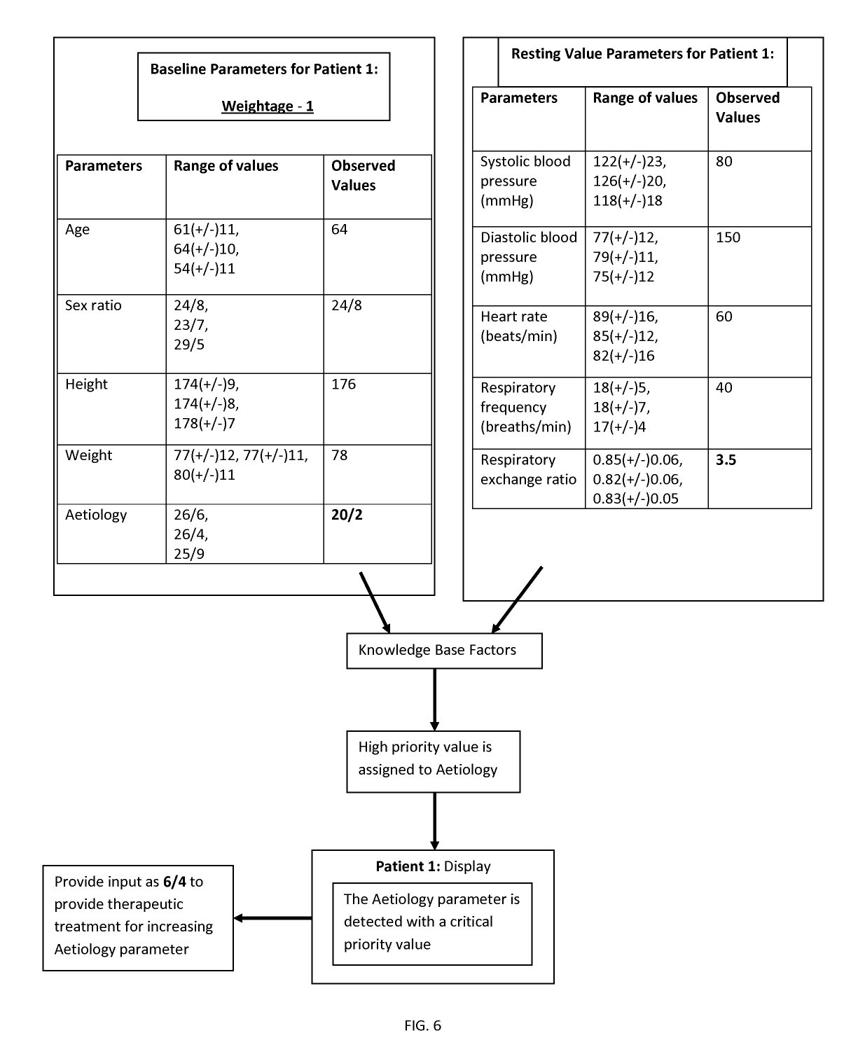

Referring to FIG. 6, two physiology parameters are considered for determining the priority of the parameter values for patient 1. The two physiology parameters that are considered are for determining the priority are Baseline parameters and Resting value parameters. As depicted in the example: the Baseline parameter is assigned a higher weightage (weightage 1) as compared to the weightage assigned to the Resting value (weightage 5) parameter. Further, the observed values are determined for each of the parameters considered under the Baseline parameter and the Resting value parameter. Further, the observed values along with the weightage assigned to each of the parameters are associated and mapped with the existing knowledge base in the HIPAA server. After associating and mapping the parameter values with the existing knowledge base in the HIPAA server, the priority value is computed. In the current example, a high priority value is determined for the Aetiology parameter. Hence, a warning message is displayed to patient 1 stating that the Aetiology is detected with a high priority value. Based on this warning message, input is provided by the user for initiating a therapeutic treatment. Here, an input value 26/6 is provided for initiating the therapeutic treatment and increasing the Aetiology parameter value to the required range.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

What is claimed is:

1. A method for initializing at least one therapeutic treatment for normalizing a high priority value computed for at least one monitored physiology data, wherein said method comprises of:
(a) computing a priority value for at least one monitored physiology data received at a Health Insurance Portability and Accountability Act (HIPAA) server;
(b) determining said at least one monitored physiology data that has said high priority value; and
(c) providing at least one input to initialize said at least one therapeutic treatment for normalizing said high priority value of said at least one monitored physiology data.

2. The method as claimed in claim 1, wherein said at least one therapeutic treatment can be initialized by providing at least one input by performing a gesture on an electronic device or by configuring at least one value for resetting said at least one monitored physiology data to a normal priority.

3. The method as claimed in claim 1, wherein said method comprises of computing priority by assigning a weightage and a range of values to at least one parameter associated with said at least one monitored physiology data for detecting criticality of said at least one patient's condition.

4. The method as claimed in claim 3, wherein said computed priority is further optimized by associating and mapping said weightage and said range of values of said at least one parameter with said knowledge base maintained on said HIPAA server.

5. The method as claimed in claim 1, wherein said method further comprises of displaying said at least one monitored physiology data that is assigned with a high priority value on said at least one electronic device.

6. The method as claimed in claim 1, wherein said method further comprises of tracking feedback for said computed priority comprises of receiving a feedback response from said at least one patient, wherein said at least one patient is notified by said computed priority of said at least one monitored physiology data.

7. The method as claimed in claim 6, wherein said computed priority of said at least one monitored physiology data must be set to a high priority value.

8. The method as claimed in claim 3, wherein said knowledge base maintained on said HIPAA server is updated with said weightage and said range of values for said at least one parameter only when a positive tracking feedback is received from said at least one patient for said prioritized monitored physiology data.

9. The method as claimed in claim 1, wherein said at least one monitored physiology data along with said computed priority is stored or upgraded on a cloud database.
